(19) United States
(12) Patent Application Publication
(10) Pub. No.: US 2013/0102471 A1
(43) Pub. Date: Apr. 25, 2013

(54) FORMULA COMPOSITION AS DELIVERY SYSTEM USED IN PHARMACEUTICAL AND PESTICIDE APPLICATIONS

(71) Applicant: Huu Tieu, Porterville, CA (US)

(72) Inventor: Huu Tieu, Porterville, CA (US)

(21) Appl. No.: 13/653,926

(22) Filed: Oct. 17, 2012

Related U.S. Application Data
(60) Provisional application No. 61/549,188, filed on Oct. 19, 2011.

(51) Int. Cl. A61K 47/06; A61K 47/20; A01N 25/02; A61K 47/10

(52) U.S. Cl. 504/362; 514/789; 514/772; 514/788

(57) ABSTRACT

The present invention is a formula composition that acts as an efficient delivery system to transport active components to their desired target sites in the bodies of human, animal, or plant species. This formula composition is comprised of d-Limonene (C10H16), Glycol EB (2-Butoxyethanol), Calimulse PRS (Benzenesulfonic acid, C10-16-alkyl derivatives, compounds with 2-propanamine), Calsoft L-60 (Benzenesulfonic acid, C10-16-alkyl derivatives, sodium salts, Sodium xylene sulfonate, sodium sulfate), Isopar M Solvent (Light Hydrotreated Petroleum Distillates), and water. This formula composition has both pharmaceutical and agricultural product applications, due to its ability to emulsify certain active components, preserve active components in their intact form, quickly deliver them to their target sites in the body, and increase their absorptions. The formula composition in the present invention performs above functions through the synergy of its components, as well as its excellent capacity of membrane transportation.

The current application claims a priority to the U.S. Provisional Patent application Ser. No. 61/549,188 filed on Oct. 19, 2011.

### FIELD OF THE INVENTION

The present invention relates to a formula composition for the purpose of efficiently providing delivery and absorption for certain active components in the Pharmaceutical and Pesticidal applications. More particularly, the invention relates to a formula composition comprising certain emulsifiers and solvents including naturally derived D-Limonene. It functions as an efficient delivery system to carry out certain active components to the target areas in the bodies of human, animal or plant species. Moreover, this formula composition is able to quickly transport such active components through the circulatory system, and maintain them in a chemically unchanged form. Hence those active components will have an almost intact bioavailability when they arrive at the target areas.

### BACKGROUND OF THE INVENTION

In the fields of medicine, pharmacy and agricultural pesticides, certain active components (medical composition and compound, drugs, pesticides et al) themselves usually cannot efficiently penetrate the cellular membranes and quickly travel to their target sites in the body; nor can they keep their activities or bioavailability during the passage. So one of the most critical challenges is to design highly efficient delivery systems that can quickly transport the active components to their target sites in the body; and meanwhile, help to maintain such active components in their active form during the process of delivery. The present invention is related to a newly-developed formula composition as an efficient delivery system that can transport certain active components to their target sites in the body, as well as preserve their activities during the transportation. This formula composition comprises multiple components. Among them, d-Limonene is a major solvent component in the formulation.

The chemical compound d-Limonene has been around for many years and proven to be safe for use with plants, animals and humans. This chemical is a major constituent of citrus oils, including those obtained from orange, lemon, lime and grapefruit, extract from citrus rinds or peels. During the process of making juice from citrus fruits, citrus oil is pressed out of the rind. This citrus oil is next separated from juice and then distilled in order to recover certain flavor and fragrant compounds from the oil. The bulk of the oil, however, is left behind and collected, which contains the food grade d-Limonene. To be more specific, after the juicing process, the rinds are conveyed to a steam extractor to extract more oil from the rind. The steam is condensed; and a layer of citrus oil floats on the surface of the condensed water. This layer of citrus oil will provide technical grade d-Limonene. It is well known that, in addition to being a very safe chemical itself, d-Limonene can also be utilized with other chemicals to generate a series of compositions that would function as fungicide, bactericide, adjuvant, or other agricultural and pharmaceutical products. For instances, in U.S. Pat. No. 3,023,144, it described certain d-Limonene based compounds configured as topical agents for human and animal use in the forms of ointments, lotions, creams, shampoos or other similar products. The U.S. Pat. No. 3,960,539 to Newhall disclosed a d-Limonene use in a compound for the regulation of plant growth and control of nematodes and fungi on plants. U.S. Pat. No. 4,379,168 to Dotolo described the use of d-Limonene in a pesticidal composition that also contains a surfactant or emulsifier in water to form a composition that is non-toxic and no-irritating to animals.

2-Butoxyethanol is an organic solvent with the formula C6H14O2. The chemical is a colorless liquid with a sweet, ether-like odor. It is butyl ether of ethylene glycol. The main use of 2-Butoxyethanol is as a solvent in paints and surface coatings, followed by in cleaning products and inks. Other products which contain 2-Butoxyethanol include acrylic resin formulation, asphalt release agents, firefighting foam, leather protectors, oil spill dispersants, bowling pin and lane degreaser, and photographic strip solutions. 2-Butoxyethanol is also a primary ingredient of various whiteboard cleaners, liquid soaps, cosmetics, dry cleaning solutions, lacquers, varnishes, latex paints, and other agricultural or pharmaceutical products. It also appears to be excellent at killing most insects and arachnids. The chemical 2-Butoxyethanol usually decomposes in the environment within a few days and is not identified as an environmental contaminant. It is not known to build up in the body of any plant or animal species either.

Calimulse-PRS (Benzenesulfonic acid, C10-16-alkyl derivatives, compounds with 2-propanamine) is produced using a proprietary sulfonation process to ensure its consistency, uniformity and high purity, which make it an excellent emulsifier being used in a list of industrial products for degreasing surfaces, promoting latex emulsification, dry cleaning, improving pigment dispersion, oil-slick emulsification, enhancing oil recovery in oil field, and other pharmaceutical or agricultural processes. Calimulse-PRS (Benzenesulfonic acid, C10-16-alkyl derivatives, compounds with 2-propanamine) is an excellent active degreaser, wetting agent and detergent. Its application also includes HI&I Biodegradable, which has been approved under the regulation of CFR21.

Calsoft-L 60 (Benzenesulfonic acid, C10-16-alkyl derivatives, sodium salts, Sodium xylene sulfonate, sodium sulfate) is a high purity, stable sulfonate used in all-purpose cleaners, detergents, degreasers, emulsifiers and other agricultural or pharmaceutical products. Manufactured by various producers with a proprietory sulfonation process; Calsoft-L 60 (Benzenesulfonic acid, C10-16-alkyl derivatives, sodium salts, Sodium xylene sulfonate, sodium sulfate) is a biodegradable, dependable, low colored uniform product with a high purity. Calsoft-L 60 (Benzenesulfonic acid, C10-16-alkyl derivatives, sodium salts, Sodium xylene sulfonate, sodium sulfate) is available as free-flowing dry material, paste liquid or in various salt forms. Calsoft-L 60 (Benzenesulfonic acid, C10-16-alkyl derivatives, sodium salts, Sodium xylene sulfonate, sodium sulfate) is an EPA and DFE approved product in the U.S. and being listed as a clean ingredient.

Isopar M Solvent (Light Hydrotreated Petroleum Distillates) is a clear isoparaffin fluid, typically containing less than 1 ppm benzene and less than 1 ppm sulphur. The Isopar M fluids are high-purity synthetic isoparaffins with a consistent and uniform quality, because of its highly controlled manufacturing processes and usage of selected feedstock in the production. It can deliver one of the narrowest boiling ranges from any hydrocarbon fluid. They are produced at the manufacturing facilities comply with the Good Laboratory Practice. The high purity of Isopar M fluid, along with an array of technical attributes, offers various end-use benefits for both industrial processes and pharmaceutical and agricultural product applications.

Water is the chemical substance with chemical formula H2O. One molecule of water has two hydrogen atoms covalently bonded to a single oxygen atom. Water is widely used in chemical reactions as a solvent or reactant, and less commonly used as a solute or catalyst. In inorganic reaction, water is a common solvent, dissolving many ionic compounds. In organic reactions, it usually is not used as a reaction solvent, because it does not dissolve the reactants well and is amphoteric (acidic and basic) and nucleophilic. These properties are sometimes desirable in many cases.

The immune system of the human body is known to be able to resist many types of diseases and “treat” diseases and other medical conditions without the addition of any medicines that are commonly utilized today and prescribed by the medical community. However, when the body cannot achieve a self reliant balance, which reduces the capacity of immune system, many pharmaceutical, holistic or herbal practices may function to boost it. In this context, the present invention aims to provide an effective delivery system that transports certain medically effective components (drug, composition, compound etc) to the parts of the human or animal body, which are in need of the treatment. In addition, in plant physiology, the present invention may also provide a useful delivery system for transporting certain chemical compounds or active components to the target sites in the plant. The plant circulatory system provides a pathway to allow the formula composition facilitate the transport of the active components to their targets, wherein the active components could be pesticides, fertilizers or other compositions used in the agriculture. Furthermore, this formula composition helps to keep the transported active components with their intact activities.

### SUMMARY OF THE INVENTION

The present invention aims to provide a formula composition that can function as an efficient delivery system to deliver certain active components to their target sites in the body of human, animal or plant. And meanwhile, the formula composition also helps to maintain such active components' activity during the passage. The formula composition in the present invention achieves above goals through the synergy of each individual component it contains.

In a general aspect of the present invention, the formula composition comprises a mixture of d-Limonene, an organic solvent as a primary component, along with one or more other solvents and emulsifiers. In a preferred embodiment of the present invention, another organic solvent component is 2-Butoxyethanol. In another preferred embodiment, the formula composition further comprises Isoparaffinic Hydrocarbon fluid as a solvent at 5% to 15% by volume; and “Benzenesulfonic acid, C10-16-alkyl derivatives, compounds with 2-propanamine” as an emulsifier at 5% to 10% by volume. The preferred configuration of the present composition may also contain “Benzenesulfonic acid, C10-16-alkyl derivatives, sodium salts, Sodium xylene sulfonate, sodium sulfate” as an additional emulsifier at 10% to 20% by volume. In yet another preferred embodiment, the d-Limonene is present in an approximate amount of 30% to 40% by volume; 2-Butoxyethanol is present in an approximate range of 20% 30% by volume; and water at 5% to 15% by volume.

Accordingly, the primary objective of the present invention is to provide a formula composition as an efficient delivery system for certain active components. It can quickly transport such active components to their targeted locations in the body, while preserving the active components intact and maintain their activities during the passage.

Another important objective of the present invention is to provide a formula composition that can be administered orally along with active components, preferably sublingually, so as to eliminate the need to utilize invasive administration techniques such as, needle injection, IV injection or other invasive techniques, though this administration may also be used when necessary. The composition may also be administered topically depending on the type and property of the active components being introduced.

Yet another objective of the present invention is to provide a formula composition comprising d-Limonene (C10H16), Glycol EB (2-Butoxyethanol), Calimulse PRS (Benzenesulfonic acid, C10-16-alkyl derivatives, compounds with 2-propanamine), Calsoft L-60 (Benzenesulfonic acid, C10-16-alkyl derivatives, sodium salts, Sodium xylene sulfonate, sodium sulfate), Isopar M Solvent (Light Hydro treated Petroleum Distillates), and Water (H2O) as the essential components. This formulation provides a unique and effective delivery system that facilitates the active component cargo quickly enter the body's circulation system and reach the target area where the active components are needed to achieve their desired results.

### DETAILED DESCRIPTION OF THE INVENTION

With reference to the various preferred embodiments of the present invention set forth in this application, these embodiments are intended to be representative of one or more ways of configuring the formula composition of the present invention. Although specific chemicals, materials, configurations and uses are set forth herein, it should be understood that a number of variations to the components described herein can be made without changing the scope and function of the present invention. For instance, although the description provided herein sets forth certain components and percentages of those components that make up the preferred embodiments of the medical composition of the present invention, those who are skilled in the art will readily understand that this is merely for purposes of simplifying the present disclosure and that the present invention is not so limited. Specifically, as will be readily appreciated by those skilled in the art, various equivalent chemicals can be utilized to make up the formula composition of the present invention.

Provided is a formula composition as a delivery system to transport active components to their target sites in the body. In a preferred embodiment, the formula composition of the present invention comprises the following components: the chemical d-Limonene, an organic solvent 2-Butoxyethanol, the solvent Isoparaffinic fluid, an emulsifier Sodium dodecylbenzene Sulfonate and a small amount of the solvent Isopropylamine. A number of processors, such as Florida Chemical Company in Winter Haven, Fla., supply d-Limonene (1-methyl-4-(methylene) cyclohexene, C10H16), which is suitable for use in the formula composition in the present invention. In another preferred composition, the 2-Butoxyethanol component is Butyl Cellusolve or Ethylene Glycol (2-Butoxyethanol) 2-Butoxy-1-ethanol, which is available from Univar Corporation in Seattle, Wash. With regard to other components, the Isoparaffinic hydrocarbons solvent is Isopar-M C12-C15 available from Exxon-Mobile Corporation in Fairfax, Virginia. The Mono Isopropylamine Dodecylbenzene Sulfonate (alkylbenzene sulfonate, amine salt) C12H25(C6H4) O3S.C3H9N emulsifier is Benzene Sulfonic Acid. Mono-C10-16-Alkly (alkyl aryl sulfonate, sodium salt) C12H25(C6H4)SO3Na Calsoft L-60 is available from Pilot Chemical Company in Cincinnati, Ohio. The Isopropylamine as Calimulse PRS is available from Pilot Chemical Company. As will be readily apparent to those skilled in the art, various equivalent chemicals and chemical compositions can be substituted for those identified above and effectively accomplish the objectives of the formula composition in the present invention.

A preferred embodiment of the formula composition in the present invention is set forth in the formulation below:

| | Approximate (%) | |
|---|---|---|
| | By Volume | |
| Chemical | Referred Range | Use |
| D-Limonene | 30-40 | Solvent |
| 2-Butoxyethnaol | 20-30 | Solvent |
| Isoparaffinic Hydrocarbon | 5-15 | Solvent |
| Benzenesulfonic acid, C₁₀-₁₆-alkyl | 5-10 | Emulsifier |
| derivatives, compounds with | | |
| 2-propanamine | | |
| Benzenesulfonic acid, C₁₀-₁₆-alkyl | 10-20 | Emulsifier |
| derivatives, sodium salts, Sodium | | |
| xylene sulfonate, sodium sulfate | | |
| Water | 5-15 | Solvent |

The unique delivery system of this formula composition in the present invention is substantially responsible for delivering active components or compounds to their target sites in the body, and protecting active components in their chemically intact form. The claimed formula composition helps to maintain the active components, such as medical composition or compound at a bioavailability (BA) of nearly 100%. Additionally, this formula composition has the capability to cross the “Blood-Brain Barrier” and enter the “Central Nervous System”, thereby being beneficial in a variety of central nervous system conditions. Besides, concerning its application with plants, the formula composition acts to facilitate the absorption and membrane transportation across the surface of plant leaves to allow the active components to penetrate into the plant's circulation system and further reach their target locations. The formula composition will also act as a protectant on the leaf surface until the surface absorption of active components has been completed.

The formula composition has a moderate reactivity in some cases when adding an active component or compound to it. Once added, a chemical reaction may occur. This could be a beneficial reaction as the molecules are binding and absorbing to create a new chemical compound or composition. Once the reaction has been completed, the new composition is achieved. Moreover, the formula composition is stable at a pH level of pH 6-7.

The formula composition of the present invention is manufactured by mixing each component in a sequence of order, beginning with d-Limonene, Isopropylamine (PRS), Sodium dodecylbenzene sulfonate (L-60), 2-Butoxyethanol (EB) and Isoparffinic fluid (Isopar). The first three components must be added in the order, and set forth at about 65° F. or above to prevent separation of the components. All the components in formula composition are tech grade and no reaction occurs when formulating and only minor resulting impurities (some settling and layering) have been noticed in a bath test and in a test conducted at three months intervals. The resulting formula composition of the present invention is far superior to the formulations that are currently available. The physical properties of the compound include having a citrus smell, a taste that is tangy and moderately bitter, is suitable for room temperature storage and has a shelf-life of approximately three years after the manufacturing date. Also it should be stored in a stainless steel container. The inherent beneficial properties of the formula composition in the present invention are achieved with natural (i.e., non-synthetic) components that are ubiquitous in nature. The benefits of the compound of the present invention are applicable to humans, animals and plants. During the mixing process, the overall volume of the composition may be reduced by approximately 2% to 5% of its mass. This synergy may help to incorporate each component, along with their beneficial effects.

While there are shown and described herein specific forms of the invention, it will be readily apparent to those skilled in the art that the invention is not so limited, but is susceptible to various modifications and rearrangements in materials without departing from the function, spirit and scope of the present invention. In particular, it should be noted that the present invention is subject to modification with regard to any of a variety of combinations of materials and the prescribed use of the formula composition. For instance, there are numerous components described herein that can be replaced with equivalent functioning components to accomplish the objectives of the present invention.

What is claimed is:

1. A formula composition for efficiently delivering active components to their target sites in the body, comprising:
one or more than one solvent, wherein at least one solvent is an organic solvent, at least one solvent is non-synthetic and derived from natural citrus plant or its fruit, and said organic solvent and said citrus-derived solvent can be the same;
one or more than one emulsifier; and
water.

2. The formula composition for efficiently delivering active components to their target sites in the body of claim 1, wherein a solvent component is D-Limonene.

3. The formula composition for efficiently delivering active components to their target sites in the body of claim 2, wherein said D-Limonene is 30% to 40% of the said formula composition by volume.

4. The formula composition for efficiently delivering active components to their target sites in the body of claim 2, further comprising an organic solvent, which is 2-Butoxyethanol.

5. The formula composition for efficiently delivering active components to their target sites in the body of claim 4, wherein said 2-Butoxyethanol is 20% to 30% of the said formula composition by volume.

6. The formula composition for efficiently delivering active components to their target sites in the body of claim 4, further comprising a solvent, which is Isoparaffinic Hydrocarbon.

7. The formula composition for efficiently delivering active components to their target sites in the body of claim 6, wherein said Isoparaffinic Hydrocarbon is 5% to 15% of the said formula composition by volume.

8. The formula composition for efficiently delivering active components to their target sites in the body of claim 6, wherein said Isoparaffinic Hydrocarbon is Isopar-M Solvent, and said Isopar-M Solvent is Light Hydro treated Petroleum Distillates.

9. The formula composition for efficiently delivering active components to their target sites in the body of claim 1, wherein said emulsifier is Calimulse PRS, which is comprised of Benzenesulfonic acid, C10-16-alkyl derivatives, compounds with 2-propanamine.

10. The formula composition for efficiently delivering active components to their target sites in the body of claim 9, further comprising another emulsifier, Calsoft L-60, which is comprised of Benzenesulfonic acid, C10-16-alkyl derivatives, sodium salts, Sodium xylene sulfonate, sodium sulfate.

11. The formula composition for efficiently delivering active components to their target sites in the body of claim 10, wherein said Calimulse PRS is 5% to 10% of the said formula composition by volume, and said Calsoft L-60 is 10% to 20% of the said formula composition by volume.

12. The formula composition for efficiently delivering active components to their target sites in the body of claim 1, wherein said formula composition is a liquid with the following properties: 1) a citrus smell, 2) a tangy and moderately bitter taste, 3) suitable for room temperature storage, and 4) with a shelf-life of three years after the manufacturing date.

13. The formula composition for efficiently delivering active components to their target sites in the body of claim 1, wherein said formula composition is stable at pH 6-7, and stored in a stainless steel container.

14. The formula composition for efficiently delivering active components to their target sites in the body of claim 1, wherein said formula composition functions to protect an active component intact during passage and maintain active component's bioavailability.

15. The formula composition for efficiently delivering active components to their target sites in the body of claim 1, wherein said formula composition is used in human or animals.

16. The formula composition for efficiently delivering active components to their target sites in the body of claim 15, wherein said formula composition has the capability to cross the “Blood-Brain Barrier” and enter the “Central Nervous System”, thereby being beneficial in a variety of central nervous system conditions.

17. The formula composition for efficiently delivering active components to their target sites in the body of claim 1, wherein said formula composition is used in plants.

18. The formula composition for efficiently delivering active components to their target sites in the body of claim 17, wherein said formula composition acts as a protectant on the leaf surface until the surface absorption of active components has been completed, and facilitates transportation across the surface of plant leaves to allow active components penetrate into the plant's circulation system and further reach their target locations.

19. The formula composition for efficiently delivering active components to their target sites in the body of claim 15, wherein said formula composition is administered orally including sublingually, or administered by needle injection, including IV injection.

20. The formula composition for efficiently delivering active components to their target sites in the body of claim 15, wherein said formula composition is administered topically.
